(19) United States
(12) Patent Application Publication
(10) Pub. No.: US 2024/0199774 A1
(43) Pub. Date: Jun. 20, 2024

(54) Cationically Charged Membranes

(71) Applicants: Fujifilm Manufacturing Europe B.V., Tilburg (NL); Fujifilm Corporation, Tokyo (JP)

(72) Inventors: Adrianus Jacobus Van Rijen, Tilburg (NL); Jacko Hessing, Tilburg (NL)

(21) Appl. No.: 18/282,820

(22) PCT Filed: Mar. 24, 2022
(86) PCT No.: PCT/EP2022/057852

§ 371 Date: Sep. 19, 2023

(30) Foreign Application Priority Data
Mar. 29, 2021 (GB) 2104408.6

(51) Int. Cl. C08F 126/06; B01D 15/36; B01D 61/44; B01D 69/02; C08F 226/06

(52) U.S. Cl. CPC C08F 126/06; B01D 15/36; B01D 61/44; B01D 69/02; C08F 226/06

(57) ABSTRACT

Cationically charged membranes obtainable from curing a composition comprising an aromatic heterocyclic compound, wherein the aromatic heterocyclic compound comprises: a) an aromatic heterocyclic ring: b) at least two polymerisable groups: and c) a cationically charged nitrogen atom. The membranes are mechanically strong, have a high charge density and maintain good permselectivity even after exposure to harsh conditions such as extremes of pH.

This invention is related to cationically charged membranes and to processes for their preparation and to their uses.

Cationically charged membranes are used in a wide variety of devices, including electrodialysis devices, reverse electrodialysis devices and fuel cells. The problem with many cationically charged membranes is that exposure to harsh conditions, e.g. high or low pH, adversely affects their permselectivity. There is a need for cationically charged membranes which are mechanically strong, have a high charge density and maintain good permselectivity even after exposure to harsh conditions such as a low and/or high pH.

According to a first aspect of the present invention there is provided a cationically charged membrane obtainable from curing a composition comprising an aromatic heterocyclic compound, wherein the aromatic heterocyclic compound comprises:
a) an aromatic heterocyclic ring;
b) at least two polymerisable groups; and
c) a cationically charged nitrogen atom.

In this specification the term “comprising” is to be interpreted as specifying the presence of the stated parts, steps or components, but does not exclude the presence of one or more additional parts, steps or components.

Reference to an element by the indefinite article “a” or “an” does not exclude the possibility that more than one of the element(s) is present, unless the context clearly requires that there be one and only one of the elements. The indefinite article “a” or “an” thus usually means “at least one”.

Preferably the aromatic heterocyclic compound has a molecular weight (MW) of less than 500 n Dalton, more preferably less than 400 n Dalton, especially less than 300 n Dalton and more especially less than 250 n Dalton, wherein n is the number of cationically charged nitrogen atoms present in the aromatic heterocyclic compound. For example, an aromatic heterocyclic compound having 2 cationically charged nitrogen atoms would preferably have a MW of less than 500*2=less than 1,000 Dalton.

Preferably cationically charged nitrogen atom c) is a part of the aromatic heterocyclic ring (a), i.e. one of the ring atoms. This preference arises because such compounds can provide membranes having a particularly high ion-exchange capacity (IEC). Without wishing to be limited to any particular theory, it is believed that the high IEC arises at least in part from the fact that the cationic charge is part of the aromatic heterocyclic ring making the molecule more compact than, for example, styrenic crosslinking agents having separate quaternary ammonium groups. By ensuring that the cationic charged nitrogen atom c) is part of the aromatic heterocyclic ring (a) makes it possible to incorporate more charges into the membrane, opening up opportunities to provide membranes having the ability to maintain good permselectivity in combination with a low electrical resistance (ER) even after being exposed to harsh conditions. Preferably the membrane has an ion exchange capacity of at least 2.4 meq/g; preferably the ion exchange capacity is less than 5.1 meq/g to prevent excessive swelling.

Preferably the aromatic heterocyclic compound comprises two, three or four aromatic rings, at least one of which is heterocyclic and at least one of which comprises a cationically charged nitrogen atom (N⁺).

The aromatic heterocyclic ring optionally further comprises an uncharged hetero-atom selected from N, O and S.

The aromatic heterocyclic ring is preferably a 5- or 6-membered aromatic heterocyclic ring. Preferred 5- and 6-membered aromatic heterocyclic rings comprise a cationically charged nitrogen atom (N⁺), three, four or five ring carbon atoms and optionally one or two ring atoms selected from O, N and S.

Preferred polymerisable groups b) comprise ethylenically unsaturated groups, thiol groups and epoxy groups. Preferred ethylenically unsaturated groups include vinyl groups, allyl groups, (meth)acrylic groups (e.g. CH₂═CR¹—C(O)— groups), especially (meth)acrylate groups (e.g. CH₂═CR¹—C(O)O— groups) and (meth)acrylamide groups (e.g. CH₂═CR¹—C(O)NR¹— groups), wherein each R¹ independently is H or CH₃). Most preferred polymerisable groups are vinyl groups (CH₂═CH—) and allyl groups (CH₂═CH—CH₂—). Preferably the vinyl groups are non-acrylic, i.e. the vinyl groups are not attached to (C═O)O-groups or (C═O)NH-groups.

Preferably the polymerisable groups b) are attached directly to the aromatic heterocyclic ring c) as this provides for fast and efficient curing to form the membrane.

As counter-ion for the cationically charged nitrogen atom any anion may be used. Preferably the anionic counterion does not react with the other components of the composition, i.e. is inert. Preferred counter-ions include hydroxide, fluoride, chloride, bromide, iodide, nitrate, thiocyanate, hexafluoroborate, methanesulfonate, trifluoromethanesulfonate, formate and acetate. Most preferred counterion is a chloride anion because compounds with a chloride counterion have a higher solubility and contribute less than other counterions to the molecular weight of the compound.

The aromatic heterocyclic ring is preferably a pyridine, thiazole, isothiazole, pyrrole, pyrazole, imidazole, triazole, pyrimidine, pyridazine, pyrazine, oxazole, cinnoline, quinazoline, quinoxaline, phthalazine, peteridine or carbazole ring.

Thus the aromatic heterocyclic compound preferably comprises at least one pyridine, thiazole, isothiazole, pyrrole, pyrazole, imidazole, triazole, pyrimidine, pyridazine, pyrazine, oxazole, cinnoline, quinazoline, quinoxaline, phthalazine, peteridine or carbazole ring which, in each case, preferably comprises a cationically charged nitrogen atom and optionally a phenyl group.

The aromatic heterocyclic compound is preferably of Formula (I):

A-Z-B Formula (I)
wherein:
A and/or B are each independently selected from aromatic heterocyclic rings of the following formulae and one of A and B is optionally an optionally substituted phenyl group:

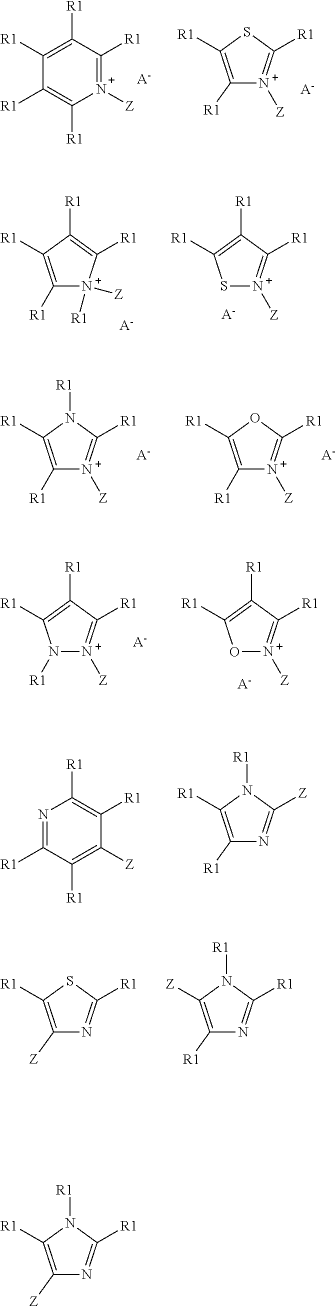

wherein:
each R1 independently is H, halogen, a polymerisable group or C₁₋₄-alkyl;
A⁻ is any negatively charged counterion (e.g. hydroxide, fluoride, chloride, bromide, iodide, hexafluoroborate, nitrate, thiocyanate, methanesulfonate, trifluoromethanesulfonate, formate, acetate).
Z is a linking group;
provided that:
(i) at least one of A and B comprises a cationically charged nitrogen atom;
(ii) A and B each comprise at least one polymerisable group.

The polymerisable group is preferably a vinyl group.

In one embodiment A and B each comprise one and only one polymerisable group.

Several of the above aromatic heterocyclic rings shown above comprise a cationically charged nitrogen atom (N⁺).

Preferably Z is selected from optionally substituted C₁-alkylene; optionally substituted C₈-₁₂-arylene, optionally substituted C₁-alkylenearylene, optionally substituted dimethylene ether, optionally substituted trimethylene amine or a combination thereof, or Z is a direct bond. Preferably Z is not a direct bond if Z connects two charged nitrogen atoms. Optional substituents include, when present, C₁₋₄-alkyl, C₁₋₄-alkoxy, ammonium and hydroxyl groups.

A or B may further comprise an aromatic non-heterocyclic ring:

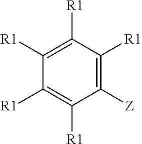

wherein each R1 independently is as defined above.

Examples of aromatic heterocyclic compounds include the following:

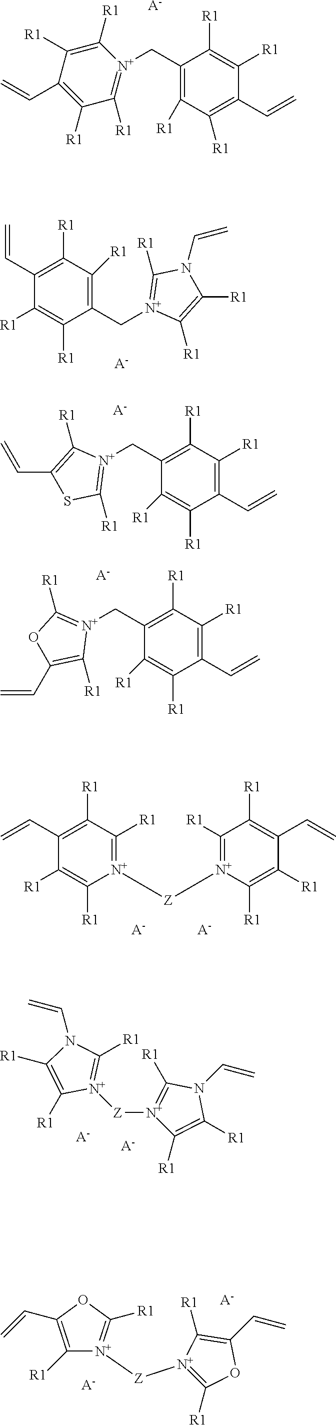

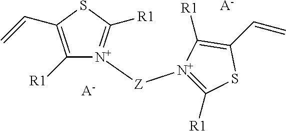

wherein each R1 and A⁻ independently are as defined above.

Specific Examples of aromatic heterocyclic compounds include the following:

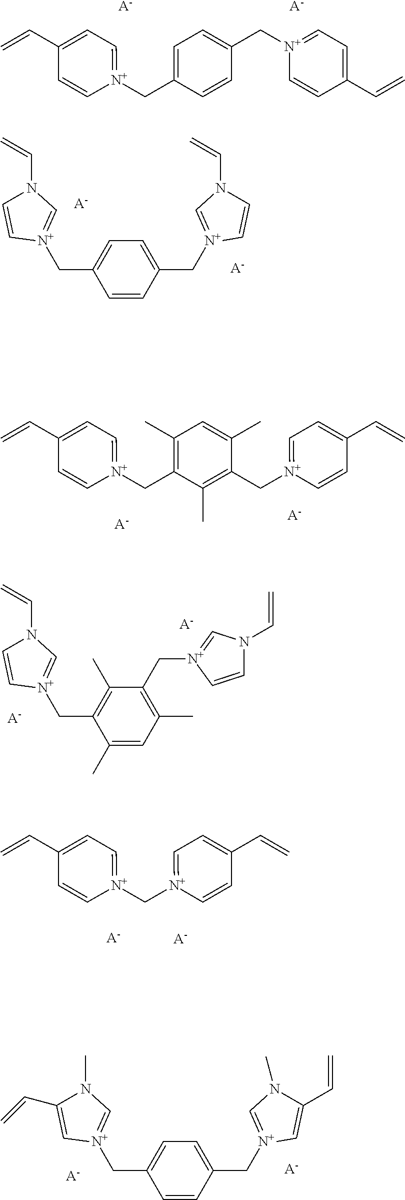

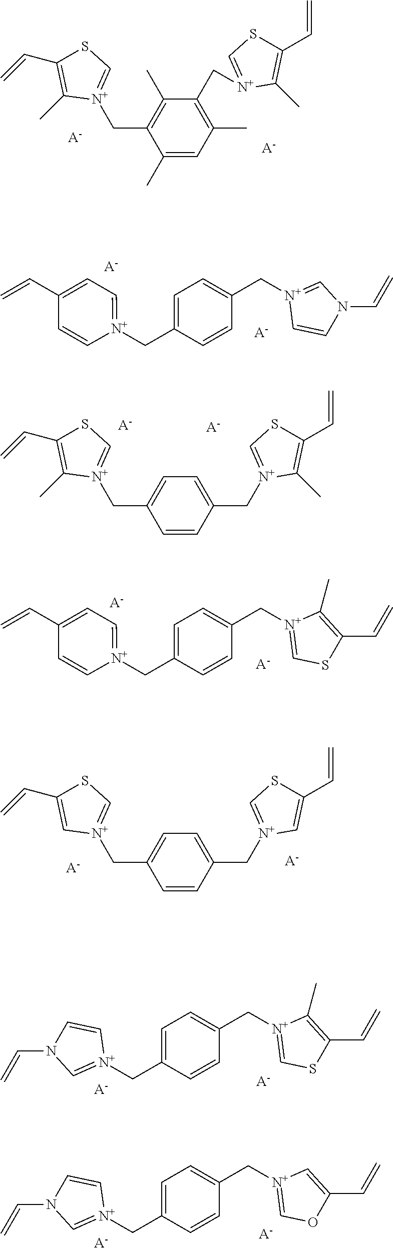

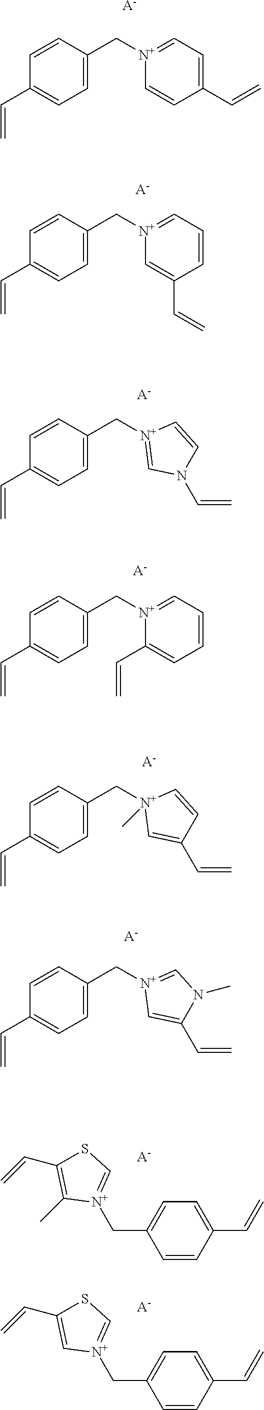

In one embodiment the aromatic heterocyclic compound comprises at least two cationically charged nitrogen atoms.

Preferably the aromatic heterocyclic compound comprises at least two cationically charged nitrogen atoms and the distance between the at least two cationically charged nitrogen atoms is at least 0.35 nm.

Preferably, the cationically charged nitrogen atom(s) of the aromatic heterocyclic compound is(are) covalently bound to either an (optionally substituted) arylene group or an (optionally substituted) alkylarylene or alkylene group of which the aliphatic parts (non-aromatic) are not larger than one carbon atom. This is to ensure that no Hoffman-elimination process can occur in high-pH environments. Preferably the charged nitrogen atom is covalently bound to a non-aromatic C₁-alkyl (methylene) group.

Preferably the cationically charged membrane further comprises a porous support.

The membrane according to the first aspect of the present invention is preferably obtainable by curing a composition comprising:
(a) the aromatic heterocyclic compound as defined above;
optionally (b) a cationically charged compound comprising only one polymerisable group;
optionally (c) one or more radical initiators;
optionally (d) one or more monomer free from cationically charged groups and
optionally (e) inert solvent.

The polymerisable group in component (b) is preferably a vinyl group.

Preferably the composition comprises one, two or all three of components (b), (c) and (d). When any of components (b), (c), (d) and/or (e) is present, the abovementioned composition forms a second aspect of the present invention.

Preferably, in some embodiments, the composition comprises 30 to 70 wt %, more preferably 35 to 60 wt %, of component (a).

Preferably the composition comprises 0 to 40 wt %, more preferably 5 to 40 wt %, most preferably 8 to 35 wt %, of component (b).

Preferably the composition comprises 0 to 10 wt %, more preferably 0.001 to 5 wt %, most preferably 0.005 to 2 wt %, of component (c).

Preferably the composition comprises 0 to 20 wt %, more preferably 0 to 12 wt %, of component (d).

Preferably the composition comprises 0 to 50 wt %, more preferably 15 to 40 wt %, most preferably 20 to 30 wt %, of component (e).

Preferably the wt % of components (a)+(b)+(c)+(d)+(e) adds up to 100 wt %. Examples of compounds which may be used as component (b) of the composition include compounds of formula (B) or (SM).

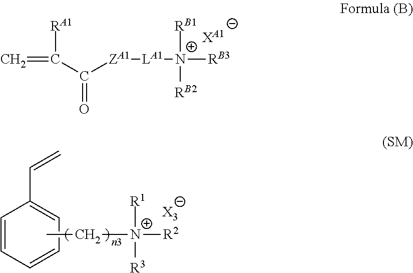

Examples of formula (B) are:

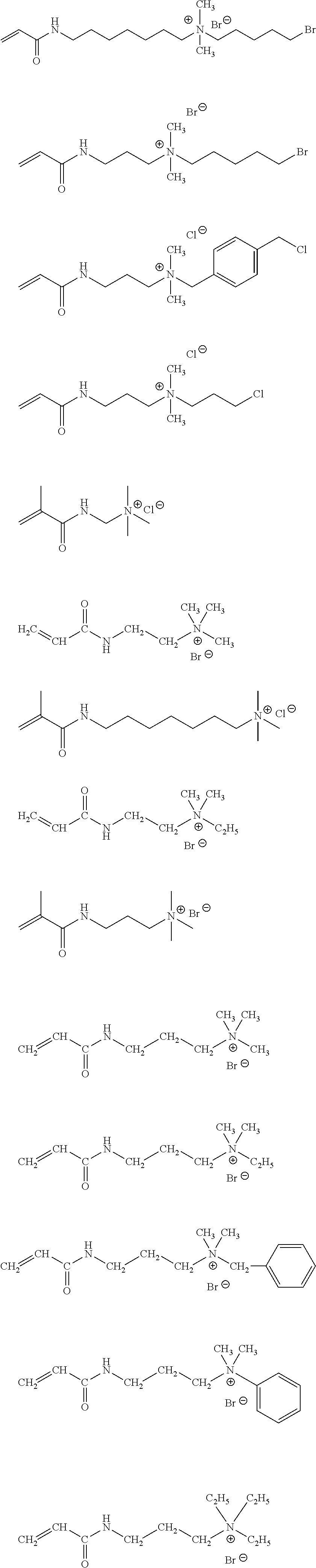

Examples of compounds of Formula (SM) are:

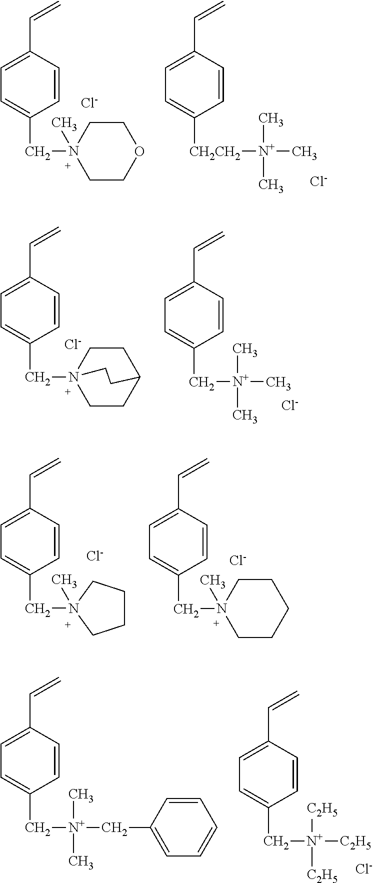

The above compounds may be prepared as described in, for example, US2016177006.

Preferably component (b) is chosen from the compounds of Formula (SM) because this can result in polymer films having especially good stability in the pH range 0 to 14.

Component (c), the radical initiator, is preferably a thermal initiator or a photoinitiator.

Examples of suitable thermal initiators which may be used as component (c) include 2,2′-azobis(2-methylpropionitrile) (AlBN), 4,4′-azobis(4-cyanovaleric acid), 2,2′-azobis(2,4-dimethyl valeronitrile), 2,2′-azobis(2-methylbutyronitrile), 1,1′-azobis(cyclohexane-1-carbonitrile), 2,2′-azobis(4-methoxy-2,4-dimethyl valeronitrile), dimethyl 2,2′-azobis(2-methylpropionate), 2,2′-azobis[N-(2-propenyl)-2-methylpropionamide, 1-[(1-cyano-1-methylethyl)azo]formamide, 2,2′-azobis(N-butyl-2-methylpropionamide), 2,2′-Azobis(N-cyclohexyl-2-methylpropionamide), 2,2′-Azobis(2-methylpropionamidine) dihydrochloride, 2,2′-azobis[2-(2-imidazolin-2-yl)propane]dihydrochloride, 2,2′-azobis[2-(2-imidazolin-2-yl)propane]disulfate dihydrate, 2,2′-azobis[N-(2-carboxyethyl)-2-methylpropionamidine] hydrate, 2,2′-azobis{2-[1-(2-hydroxyethyl)-2-imidazolin-2-yl]propane} dihydrochloride, 2,2′-azobis[2-(2-imidazolin-2-yl)propane], 2,2′-azobis(1-imino-1-pyrrolidino-2-ethylpropane) dihydrochloride, 2,2′-azobis{2-methyl-N-[1,1-bis(hydroxymethyl)-2-hydroxyethl]propionamide} and 2,2′-azobis[2-methyl-N-(2-hydroxyethyl) propionamide].

Examples of suitable photoinitiators which may be included in the composition as component (c) include aromatic ketones, acylphosphine compounds, aromatic onium salt compounds, organic peroxides, thio compounds, hexa-arylbiimidazole compounds, ketoxime ester compounds, borate compounds, azinium compounds, metallocene compounds, active ester compounds, compounds having a carbon halogen bond, and an alkyl amine compounds. Preferred examples of the aromatic ketones, the acylphosphine oxide compound, and the thio-compound include compounds having a benzophenone skeleton or a thioxanthone skeleton described in “RADIATION CURING IN POLYMER SCIENCE AND TECHNOLOGY”, pp. 77-117 (1993). More preferred examples thereof include an alpha-thiobenzophenone compound described in JP1972-6416B (JP-S47-6416B), a benzoin ether compound described in JP1972-3981B (JP-S47-3981B), an alpha-substituted benzoin compound described in JP1972-22326B (JP-S47-22326B), a benzoin derivative described in JP1972-23664B (JP-S47-23664B), an aroylphosphonic acid ester described in JP1982-30704A (JP-S57-30704A), dialkoxybenzophenone described in JP1985-26483B (JP-S60-26483B), benzoin ethers described in JP1985-26403B (JP-S60-26403B) and JP1987-81345A (JPS62-81345A), alpha-amino benzophenones described in JP1989-34242B (JP H01-34242B), U.S. Pat. No. 4,318,791A, and EP0284561A1, p-di(dimethylaminobenzoyl) benzene described in JP1990-211452A (JP-H02-211452A), a thio substituted aromatic ketone described in JP1986-194062A (JPS61-194062A), an acylphosphine sulfide described in JP1990-9597B (JP-H02-9597B), an acylphosphine described in JP1990-9596B (JP-H02-9596B), thioxanthones described in JP1988-61950B (JP-S63-61950B), and coumarins described in JP1984-42864B (JP-S59-42864B). In addition, the photoinitiators described in JP2008-105379A and JP2009-114290A are also preferable. In addition, photoinitiators described in pp. 65 to 148 of “Ultraviolet Curing System” written by Kato Kiyomi (published by Research Center Co., Ltd., 1989) may be used.

Especially preferred photoinitiators include Norrish Type II photoinitiators having an absorption maximum at a wavelength longer than 380 nm, when measured in one or more of the following solvents at a temperature of 23° C.: water, ethanol and toluene. Examples include a xanthene, flavin, curcumin, porphyrin, anthraquinone, phenoxazine, camphorquinone, phenazine, acridine, phenothiazine, xanthone, thioxanthone, thioxanthene, acridone, flavone, coumarin, fluorenone, quinoline, quinolone, naphtaquinone, quinolinone, arylmethane, azo, benzophenone, carotenoid, cyanine, phtalocyanine, dipyrrin, squarine, stilbene, styryl, triazine or anthocyanin-derived photoinitiator.

Component (d) is preferably divinylbenzene, triallylamine or a polybutadiene.

Preferably component (e) of the composition is an inert solvent. In other words, preferably component (e) does not react with any of the other components of the composition. In one embodiment the component (e) preferably comprises water and optionally an organic solvent, especially where some or all of the organic solvent is water-miscible. The water is useful for dissolving component (a), (b) and possibly also component (c) and the organic solvent is useful for dissolving component (d) or any other organic components present in the composition.

Component (e) is useful for reducing the viscosity and/or surface tension of the composition. In some embodiments, the composition comprises 15 to 40 wt %, especially 20 to 30 wt %, of component (e).

Examples of inert solvents which may be used as or in component (e) include water, alcohol-based solvents, ether based solvents, amide-based solvents, ketone-based solvents, sulphoxide-based solvents, sulphone-based solvents, nitrile-based solvents and organic phosphorus based solvents. Examples of alcohol-based solvents which may be used as or in component (e) (especially in combination with water) include methanol, ethanol, isopropanol, n-butanol, ethylene glycol, propylene glycol, diethylene glycol, dipropylene glycol and mixtures comprising two or more thereof. In addition, preferred inert, organic solvents which may be used in component (e) include dimethyl sulphoxide, dimethyl imidazolidinone, sulpholane, N-methylpyrrolidone, dimethyl formamide, acetonitrile, acetone, 1,4-dioxane, 1,3-dioxolane, tetramethyl urea, hexamethyl phosphoramide, hexamethyl phosphorotriamide, pyridine, propionitrile, butanone, cyclohexanone, tetrahydrofuran, tetrahydropyran, 2-methyltetrahydrofuran, ethylene glycol diacetate, cyclopentylmethylether, methylethylketone, ethyl acetate, y-butyrolactone and mixtures comprising two or more thereof. Dimethyl sulphoxide, N-methyl pyrrolidone, dimethyl formamide, imidazolidinone, sulpholane, acetone, dimethyl cyclopentylmethylether, methylethylketone, acetonitrile, 2-tetrahydrofuran, methyltetrahydrofuran and mixtures comprising two or more thereof are preferable.

Preferably components (a), (b) and (d) can polymerise by radiation, thermal or electron beam initiation.

According to a third aspect of the present invention there is provided a process for preparing the cationically charged membrane according to the first aspect of the present invention comprising curing a composition according to the second aspect of the present invention.

The process for preparing the cationically charged membrane preferably comprises the steps of:
i. providing a porous support;
ii. impregnating the porous support with the composition of the second aspect of the present invention; and
iii. curing the curable composition.

The preferences for the composition used in the process of the third aspect of the present invention are as described herein in relation to the second aspect of the present invention.

The compositions may be cured by any suitable process, including thermal curing, photocuring, electron beam (EB) irradiation, gamma irradiation, and combinations of the foregoing.

Preferably the process according to the third aspect of the present invention comprises a first curing step and a second curing step (dual curing). In a preferred embodiment the compositions are cured first by photocuring, e.g. by irradiating the compositions by ultraviolet or visible light, or by gamma or electron beam radiation, and thereby causing the curable components present in the compositions to polymerise, and then applying a second curing step. The second curing step preferably comprises thermal curing, gamma irradiation or EB irradiation whereby the second curing step preferably applies a different method than the first curing step. When gamma or electron beam irradiation is used in the first curing step preferably a dose of 60 to 120 kGy, more preferably a dose of 80 to 100 kGy.

In one embodiment the process according to the third aspect of the present invention comprises curing the composition in the first curing step to form the cationically charged membrane, winding the cationically charged membrane onto a core (optionally together with an inert polymer foil) and then performing the second curing step.

In a preferred embodiment the first and second curing steps are respectively selected from (i) UV curing then thermal curing; (ii) UV curing then electron beam curing; and (iii) electron beam curing then thermal curing.

The composition preferably comprises 0.05 to 5 wt % of component (d) for the first curing step. The composition optionally further comprises 0 to 5 wt % of a second component (d) for the second curing step. When it is intended to cure the composition thermally or using light (e.g. UV or visible light) the composition preferably comprises 0.001 to 2 wt %, depending on the selected radical initiator, in some embodiments 0.005 to 0.9 wt %, of component (d). Component (d) may comprise more than one radical initiator, e.g. a mixture of several photoinitiators (for single curing) or a mixture of photoinitiators and thermal initiators (for dual curing). Alternatively a second curing step is performed using gamma or EB irradiation. For the second curing step by gamma or EB irradiation preferably a dose of 20 to 100 kGy is applied, more preferably a dose of 40 to 80 kGy.

For the optional second curing step, thermal curing is preferred. The thermal curing is preferably performed at a temperature between 50 and 100° C., more preferably between 60 and 90° C. The thermal curing is preferably performed for a period between 2 and 48 hours, e.g. between 8 and 16 hours, e.g. about 10 hours. Optionally after the first curing step a polymer foil is applied to the cationically charged membrane before winding (this reduces oxygen inhibition and/or sticking of the cationically charged membrane onto itself).

Preferably the process according to the third aspect of the present invention is performed in the presence of a porous support. For example, the composition according to the second aspect of the present invention is present in and/or on a porous support. The porous support provides mechanical strength to the cationically charged membrane resulting from curing the composition according to the second aspect of the present invention and this is particularly useful when the cationically charged membrane is intended for use as an AEM or BPM.

As examples of porous supports which may be used there may be mentioned woven and non-woven synthetic fabrics and extruded films. Examples include wetlaid and drylaid non-woven material, spunbond and meltblown fabrics and nanofiber webs made from, e.g. polyethylene, polypropylene, polyacrylonitrile, polyvinyl chloride, polyphenylenesulfide, polyester, polyamide, polyaryletherketones such as polyether ether ketone and copolymers thereof. Porous supports may also be porous membranes, e.g. polysulphone, polyethersulphone, polyphenylenesulphone, polyphenylenesulfide, polyimide, polyethermide, polyamide, polyamideimide, polyacrylonitrile, polycarbonate, polyacrylate, cellulose acetate, polypropylene, poly(4-methyl 1-pentene), polyinylidene fluoride, polytetrafluoroethylene, polyhexafluoropropylene and polychlorotrifluoroethylene membranes and derivatives thereof.

The porous support preferably has an average thickness of between 10 and 800 μm, more preferably between 15 and 300 μm, especially between 20 and 150 μm, more especially between 30 and 130 μm, e.g. around 60 μm or around 100 μm.

Preferably the porous support has a porosity of 30 and 95%. The porosity of the support may be determined by a porometer, e.g. a Porolux™ 1000 from IB-FT GmbH, Germany.

The porous support, when present, may be treated to modify its surface energy, e.g. to values above 45 mN/m, preferably above 55 mN/m. Suitable treatments include corona discharge treatment, plasma glow discharge treatment, flame treatment, ultraviolet light irradiation treatment, chemical treatment or the like, e.g. for the purpose of improving the wettability of and the adhesiveness to the porous support to the cationically charged membrane.

Commercially available porous supports are available from a number of sources, e.g. from Freudenberg Filtration Technologies (Novatexx materials), Lydall Performance Materials, Celgard LLC, APorous Inc., SWM (Conwed Plastics, DelStar Technologies), Teijin, Hirose, Mitsubishi Paper Mills Ltd and Sefar AG.

Preferably the porous support is a porous polymeric support. Preferably the porous support is a woven or non-woven synthetic fabric or an extruded film without covalently bound ionic groups.

In a preferred process according to the third aspect of the present invention, the composition according to the second aspect of the present invention may be applied continuously to a moving (porous) support, preferably by means of a manufacturing unit comprising a composition application station, one or more irradiation source(s) for curing the composition, a membrane collecting station and a means for moving the support from the composition application station to the irradiation source(s) and to the membrane collecting station.

The composition application station may be located at an upstream position relative to the irradiation source(s) and the irradiation source(s) is/are located at an upstream position relative to the membrane collecting station.

Examples of suitable coating techniques for applying the composition according to the second aspect of the present invention to a porous support include slot die coating, slide coating, air knife coating, roller coating, screen-printing, and dipping. Depending on the used technique and the desired end specifications, it might be desirable to remove excess coating from the substrate by, for example, roll-to-roll squeeze, roll-to-blade or blade-to-roll squeeze, blade-to-blade squeeze or removal using coating bars. Curing by light is preferably done for the first curing step, preferably at a wavelength between 300 nm and 800 nm using a dose between 40 and 20000 mJ/cm². In some cases additional drying might be needed for which temperatures between 40° C. and 200° C. could be employed. When gamma or EB curing is used irradiation may take place under low oxygen conditions, e.g. below 200 ppm oxygen.

Preferably the cationically charged membrane is an anion exchange membrane (AEM) or an anion exchange layer (AEL) forming a part of a bipolar membrane (BPM) obtained from polymerising the composition according to the second aspect of the present invention, and/or by a process according to the third aspect of the present invention. Preferably the BPM further comprises a cation exchange layer (CEL).

Generally the cationically charged membrane comprises at least 1 ppm of the aromatic heterocyclic compound, i.e. some amount of monomer is remaining in the membrane after the curing.

According to a fourth aspect of the present invention there is provided a bipolar membrane (BPM) comprising the cationically charged membrane according to the first aspect of the present invention.

The process according to the third aspect of the present invention may be used to prepare BPMs according to the fourth aspect of the present invention in several ways, including multi-pass and single-pass processes. For example, in a two-pass process, each of the two BPM layers (the CEL and AEL) may be produced in separate steps. In the first step to make a first layer, an optionally pre-treated porous support may be impregnated with a first composition. To ensure a thin and pinhole-free membrane, the coating step is preferably followed by squeezing. The impregnated support may then be cured, yielding a layer hard enough to be handled in the coating machine, but still containing enough unreacted polymerisable groups to ensure good adhesion to the second layer. In the second step, a very similar process as for the first layer is employed: an optionally pre-treated porous support may be impregnated with a second composition and laminated to the first layer followed by squeezing-off excess composition and curing. Preferably one of the first and the second composition is the composition according to the second aspect of the present invention.

In an alternative method for making a BPM, the second layer may be coated on the first layer, followed by laminating an optionally pre-treated porous support at the side of the second composition whereby the second composition impregnates the porous support. The resulting laminate may be squeezed and cured to yield the composite membrane.

If the first composition applied in this process is the cation exchange layer (CEL), the optionally present polymer foil is removed before laminating the CEL with the anion exchange layer (AEL) and then optionally reapplied before performing the second curing step, e.g. when thermal curing is applied as second curing step.

In a more preferred single-pass process for preparing a BPM, two optionally pre-treated porous supports are unwound and each is impregnated with a composition simultaneously, wherein one of the compositions is as defined in the second aspect of the present invention to give an AEL, and the other composition comprises at least one anionic curable monomer to provide a CEL. The two layers (AEL from the composition according to the second aspect of the present invention and the CEL from the other composition) are then laminated together and squeezed, followed by curing of the resulting laminate to yield the BPM. Optionally, subsequently a second curing step is applied as described above.

The efficiency of the BPM according to the fourth aspect of the present invention may be enhanced by enlarging the surface area between the AEL and the CEL, e.g. by physical treatment (roughening) or by other means.

In one embodiment, the BPM according to the fourth aspect of the present invention optionally comprises a catalyst, e.g. metal salts, metal oxides, organometallic compounds, monomers, polymers or co-polymers or salt, preferably at the interface of the BPM's CEL and AEL.

Suitable inorganic compounds or salts which may be used as a catalyst include cations selected from, for example, group 1a through to group 4a, inclusive, together with the lanthanides and actinides, in the periodic table of elements, for example thorium, zirconium, iron, lanthanum, cobalt, cadmium, manganese, cerium, molybdenum, nickel, copper, chromium, ruthenium, rhodium, stannous, titanium and indium. Suitable salts which may be used as a catalyst include anions such as tetraborate, metaborate, silicate, metasilicate, tungstate, chlorate, phosphate, sulfate, chromate, hydroxyl, carbonate, molybdate, chloroplatinate, chloropaladite, orthovandate, tellurate and others, or mixtures of the above.

Other examples of inorganic compounds or salts which may be used as a catalyst include, but are not limited to, FeCl₃, FeCl₂, AlCl₃, MgCl₂, RuCl₃, CrCl₃, Fe(OH)₃, Al₂O₃, NiO, Zr(HPO₄)₂, MoS₂, graphene oxide, Fe-polyvinyl alcohol complexes, polyvinyl alcohol (PVA), polyethylene glycol (PEG), polyethyleneimine (PEI), polyacrylic acid (PAA), co-polymer of acrylic acid and maleic anhydride (PAAMA) and hyperbranched aliphatic polyester.

The cationically charged membrane according the present invention preferably may have a very high density as a result of preparing the cationically charged membrane from a composition according to the second aspect of the present invention having a low amount of component (e) Thus the present invention enables the production of cationically charged membrane s (e.g. AEMs and BPMs) having a very high ion exchange capacity and therefore low electrical resistance.

The cationically charged membranes and the BPMs containing an anion exchange layer (AEL) according to the present invention have good pH stability and low electrical resistance. As a result, the cationically charged membranes and BPMs according to the present invention can be used in bipolar electrodialysis to provide high voltages at low current densities. Thus when the BPMs of the present invention are used in bipolar electrodialysis processes for the production of acid and base they can provide low energy costs and/or high productivity.

According to a fifth aspect of the present invention there is provided use of the anion exchange membrane and/or the bipolar membrane according to present invention for the treatment of polar liquids, for the production the acids and bases or for the generation of electricity.

According to a sixth aspect of the present invention there is provided an electrodialysis or reverse electrodialysis unit, an electrodeionization module, a flow through capacitor, a diffusion dialysis apparatus, a membrane distillation module, an electrolyser, a redox flow battery, an acid-base flow battery or a fuel cell, comprising one or more cationically charged membranes according to the first aspect of the present invention

In the following non-limiting examples all parts and percentages are by weight unless specified otherwise.

The following analysis methods were used.

### Determination of Distance Between Cationically Charged Nitrogen Atoms within an Aromatic Heterocyclic Compound

The distance between cationically charged nitrogen atoms within an aromatic heterocyclic compound was determined by simulation using the open-source Avogadro software version 1.2.0 (see Marcus D Hanwell, Donald E Curtis, David C Lonie, Tim Vandermeersch, Eva Zurek and Geoffrey R Hutchison; “Avogadro: An advanced semantic chemical editor, visualization, and analysis platform” Journal of Cheminformatics 2012, 4:17). The structures of the aromatic heterocyclic compounds were drawn in the software and by using the auto-optimization tool the optimal chemical structure was determined. The auto-optimization tool was run with the following settings:
Force field: UFF
Steps per update: 4
Algorithm: Molecular Dynamics (300K)
No atoms were fixed or ignored

When the auto-optimization tool was finished (dE=0), the core-to-core distance between the cationically charged nitrogen atoms was determined using the ‘click to measure’ tool.

### Measurement of Electrical Resistance (ER)

ER (ohm·cm²) of the cationically charged membranes prepared in the Examples was measured by the method described by Dlugolecki et al., J. of Membrane Science, 319 (2008) on page 217-218 with the following modifications:
the auxiliary membranes were CMX and AMX from Tokuyama Soda, Japan:
the capillaries as well as the Ag/AgCl references electrodes (Metrohm type 6.0750.100) contained 3M KCl;
the calibration liquid and the liquid in compartment 2, 3, 4 and 5 was 0.5 M NaCl solution at 25° C.;
the effective membrane area was 9.62 cm²;
the distance between the capillaries was 5.0 mm;
the measuring temperature was 25° C.;
a Cole Parmer Masterflex console drive (77521-47) with easy load II model 77200-62 gear pumps was used for all compartments;
the flowrate of each stream was 475 ml/min controlled by Porter Instrument flowmeters (type 150AV-B250-4RVS) and Cole Parmer flowmeters (type G-30217-90); and
the samples were equilibrated for at least 1 hour at room temperature in a 0.5 M solution of NaCl prior to measurement.

### Measurement of Permselectivity (PS)

The permselectivity PS (%) that is the selectivity to the passage of ions of opposite charge to that of the cationically charged membranes prepared in the examples, was measured as follows. The membrane to be analysed was placed in a two-compartment system. One compartment is filled with a 0.05M solution of NaCl and the other with a 0.5M solution of NaCl.

### Settings

the capillaries as well as the Ag/AgCl reference electrodes (Metrohm type 6.0750.100) contained 3M KCl;
the effective membrane area was 9.62 cm²;
the distance between the capillaries was ca 15 mm;
the measuring temperature was 21.0±0.2° C.;
a Cole Parmer Masterflex console drive (77521-47) with easy load II model 77200-62 gear pumps was used for the two compartments;
Porter Instrument flowmeters (type 150AV-B250-4RVS) and Cole Parmer flowmeters (type G-30217-90) were used to control the flow constant at 500 ml/min;
The samples were equilibrated for 1 hr in a 0.5M NaCl solution prior to measurement. The voltage was read from a regular VOM (multitester) after 20 minutes.

Preferably the PS for NaCl is at least 85%.

### pH Stability Test

Stability of the membranes was tested by immersing a sample of the membrane under test in 4M of HCl or NaOH at 80 degrees for 7 days. After this treatment, the PS was measured and compared to the PS before the immersion. A membrane was deemed to be “OK” if, after the immersion its PS was at least 80% its original PS.

The materials shown in Table 1 were used in the Examples:

TABLE 1

| Compound | Component type | Supplier | Abbreviation |
|---|---|---|---|
| 4-vinylpyridine | Synthesis reagent | Sigma Aldrich | — |
| 4-vinylbenzyl chloride | Synthesis reagent | Sigma Aldrich | VBC |
| 2,4-bis(chloromethyl)- | Synthesis reagent | Sigma Aldrich | — |
| 1,3,5-trimethylbenzene | | | |
| Isopropylalcohol | Inert solvent | Sigma Aldrich | IPA |
| 4-hydroxy-2,2,6,6- | Polymerization | Sigma Aldrich | 4-OH- |
| tetramethylpiperidin-1- | inhibitor | | TEMPO |
| oxyl | | | |
| methylethylketone | Synthesis solvent | Sigma Aldrich | MEK |
| n-vinyl imidazole | Synthesis reagent | Sigma Aldrich | — |
| acetonitrile | Synthesis solvent | Sigma Aldrich | AcN |
| α,α′-Dichloro-p- | Synthesis reagent | Sigma Aldrich | — |
| xylene | | | |
| chloroform | Synthesis solvent | Sigma Aldrich | CHCl3 |
| deuterium oxide | NMR solvent | Sigma Aldrich | D₂O |
| diethylether | Synthesis solvent | Sigma Aldrich | ether |
| ethyl acetate | Synthesis solvent | Sigma Aldrich | EtOAc |
| 4-methyl-5- | Synthesis reagent | Sigma-Aldrich | — |
| vinylthiazole | | | |
| n-butylacetate | Synthesis solvent | Sigma-Aldrich | BuOAc |
| (ethyl(2,4,6- | Photoinitiator | IGM Resins | TPO-L |
| trimethylbenzoyl)- | | | |
| phenyl phosphinate | | | |
| 2-Hydroxy-2-methyl- | Photoinitiator | BASF | 1173 |
| 1-phenyl-propan-1-one | | | |
| (vinylbenzyl)trimethyl | Monofunctional | Sigma Aldrich | VBTMAC |
| ammonium chloride | monomer | | |
| FO2223-10 | Porous support | Freudenberg | — |
| Pure water | Inert solvent | | PW |
| 1% 4-OH-TEMPO in | inhibitor | Sigma Aldrich | TEMPO |
| pure water | | | |
| Dibromomethane | Synthesis reagent | Sigma Aldrich | — |
| Lewatit monoplus M | Synthesis reagent | Sigma Aldrich | Cl-exchange |
| 500 chloride form | | | resin |
| Methanol | Synthesis solvent | Sigma Aldrich | MeOH |

### EXAMPLES 1 TO 7 AND COMPARATIVE EXAMPLE 1

### Stage 1—Preparation of the Aromatic Heterocyclic Compound Comprising a) an Aromatic Heterocyclic Ring; b) At Least Two Polymerisable Groups; and c) a Cationically Charged Nitrogen Atom

### Preparation of Aromatic Heterocyclic Compound XL1

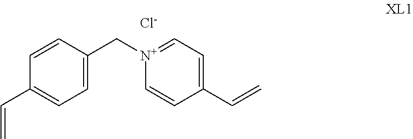 [SMILES: [Cl-] ; C=Cc1ccc(C[n+]2ccc(C=C)cc2)cc1]

4-vinylpyridine (10.5 g) and 4-vinylbenzyl chloride (15.3 g) were dissolved in isopropylalcohol (100 ml). 4-OH-TEMPO (0.1 g) was added and the mixture was heated to 65° C. and maintained at this temperature with stirring for 16 hours. The compound XL1 was precipitated from the mixture by adding methylethylketone (10 ml for every 1 ml of reaction mixture). The product, XL1, was filtered off and dried in a vacuum oven (21 g).

### Preparation of XL2

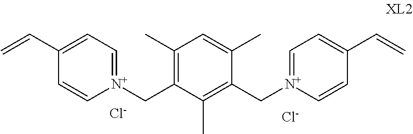 [SMILES: C=Cc1cc[n+](Cc2c(C)cc(C)c(C[n+]3ccc(C=C)cc3)c2C)cc1 ; [Cl-] ; [Cl-]]

4-vinylpyridine (21 g) and of 2,4-Bis(chloromethyl)-1,3,5-trimethylbenzene (21.7 g) were dissolved in isopropylalcohol (100 ml). 4-OH-TEMPO (0.1 g) was added and the mixture was heated to 65° C. and maintained at this temperature with stirring for 16 hours. The compound XL2 was precipitated from the mixture by adding methylethylketone (10 ml for every 1 ml of reaction mixture). The product, XL2, was filtered off and dried in a vacuum oven (25 g).

### Preparation of XL3

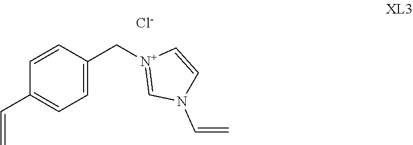 [SMILES: [Cl-] ; C=Cc1ccc(C[n+]2ccn(C=C)c2)cc1]

n-vinyl imidazole (9.4 g) and 4-vinylbenzyl chloride (15.3 g) were dissolved in acetonitrile (100 ml). 4-OH-TEMPO (0.1 g) was added and the mixture was heated to 70° C. and maintained at this temperature with stirring for 72 hours. The compound XL3 was precipitated from the mixture by adding ethyl acetate (10 ml for every 1 ml of reaction mixture). The product, XL3, was filtered off and dried in a vacuum oven (18 g).

### Preparation of XL4

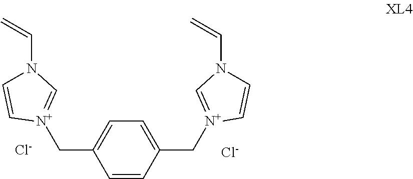 [SMILES: C=Cn1cc[n+](Cc2ccc(C[n+]3ccn(C=C)c3)cc2)c1 ; [Cl-] ; [Cl-]]

n-vinyl imidazole (18.8 g) and α,α′-dichloro-p-xylene (17.5 g) were dissolved in chloroform (100 ml). 4-OH-TEMPO (0.1 g) was added and the mixture was heated to 60° ° C. and maintained at this temperature with stirring for 72 hours. The compound XL4 was precipitated from the mixture by adding diethylether (10 ml for every 1 ml of reaction mixture). The product, XL4, was filtered off and dried in a vacuum oven (15 g).

### Preparation of XL5

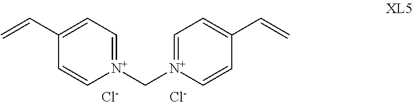 [SMILES: C=Cc1cc[n+](C[n+]2ccc(C=C)cc2)cc1 ; [Cl-] ; [Cl-]]

4-vinylpyridine (21 g) and dibromomethane (17.4 g) were dissolved in acetonitrile (100 ml). 4-OH-TEMPO (0.1 g) was added and the mixture was heated to 70° C. and maintained at this temperature with stirring for 48 hours. The counter-ion was switched from bromide to chloride by adding 100 g of Cl-exchange resin and 100 mL of MeOH. The suspension was stirred overnight at room temperature. The Cl-exchange resin was filtered off and the compound XL5 was precipitated from the mixture by adding methylethylketone (10 ml for every 1 ml of reaction mixture). The product, XL5, was filtered off and dried in a vacuum oven (12 g).

### Preparation of XL-6

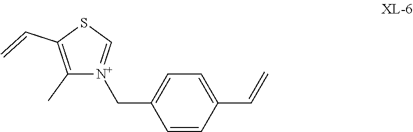 [SMILES: C=Cc1ccc(C[n+]2csc(C=C)c2C)cc1]

4-methyl-5-vinylthiazole (25.0 g) and 4-vinylbenzylchloride (30.5 g) were dissolved in 2-propanol (100 mL). 4-OH-TEMPO (0.1 g) was added and the mixture was heated to 70° C. and maintained at this temperature with stirring for 24 hours. The mixture was cooled down and XL-6 was precipitated from the mixture by adding 1200 mL n-butylacetate. The product was filtered off, washed with 100 mL n-butylacetate and dried in a vacuum oven resulting in a brown product (13 g).

### Preparation of AXL-1 (Comparative Example)

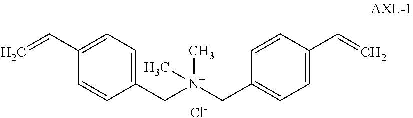 [SMILES: C=Cc1ccc(C[N+](C)(C)Cc2ccc(C=C)cc2)cc1 ; [Cl-]]

n,n-dimethyl-n-4-vinylbenzylamine (16.1 g) and 4-vinylbenzyl chloride (15.3 g) were dissolved in isopropylalcohol (100 ml). 4-OH-TEMPO (0.1 g) was added and the mixture was heated to 60° C. and maintained at this temperature with stirring for 16 hours. The compound AXL-1 was precipitated from the mixture by adding methylethylketone (10 ml for every 1 ml of reaction mixture). The product, AXL-1, was filtered off and dried in a vacuum oven (25 g).

The compositions shown in Table 2 below were prepared by mixing the stated amounts (in wt %) of the stated ingredients. Cationically charged membranes (anion exchange membranes) according to the first aspect of the present invention and Comparative Example were prepared by applying each of the compositions described in Table 2 onto a porous support (FO2223-10) using a 100 μm Meyer bar, removing the excess using a 4 μm Meyer bar and then curing the composition. UV curing was performed by placing the samples of the supports comprising the compositions on a conveyor at 5 m/min equipped with a D bulb in a Light Hammer® 10 of Fusion UV Systems Inc. and exposing the samples to the UV light emitted from the D bulb at 100% power.

The properties of the obtained cationically charged membranes are also shown in Table 2 below:

TABLE 2
Compositions and Cationically Charged Membranes:

| Materials (wt %) | Component | CEx. 1 | Ex. 1 | Ex. 2 | Ex. 3 | Ex. 4 | Ex. 5 | Ex. 6 | Ex. 7 |
|---|---|---|---|---|---|---|---|---|---|
| AXL-1 | Comparative | 49.2 | | | | | | | |
| XL-1 | (a) | | 46.4 | | | | | | 65.2 |
| XL-2 | (a) | | | 52.4 | | | | | |
| XL-3 | (a) | | | | 44.0 | | | | |
| XL-4 | (a) | | | | | 50.8 | | | |
| XL-5 | (a) | | | | | | 48.0 | | |
| XL-6 | (a) | | | | | | | 47.8 | |
| VBTMAC | (b) | 16.0 | 18.8 | 12.8 | 21.2 | 14.4 | 17.2 | 17.4 | 0 |
| TPO-L | (c) | 0.2 | 0.2 | 0.2 | 0.2 | 0.2 | 0.2 | 0.2 | 0.2 |
| 1173 | (c) | 0.3 | 0.3 | 0.3 | 0.3 | 0.3 | 0.3 | 0.3 | 0.3 |
| PW | (e) | 27.3 | 27.3 | 27.3 | 27.3 | 27.3 | 27.3 | 27.3 | 27.3 |
| IPA | (e) | 6 | 6 | 6 | 6 | 6 | 6 | 6 | 6 |
| TEMPO | | 1 | 1 | 1 | 1 | 1 | 1 | 1 | 1 |
| Number of | | 1 | 1 | 2 | 1 | 2 | 2 | 1 | 1 |
| cationically | | | | | | | | | |
| charged nitrogen | | | | | | | | | |
| atoms in | | | | | | | | | |
| component (a) (n) | | | | | | | | | |
| MW of component | | 313.9 | 257.8 | 427.4 | 246.7 | 363.3 | 295.2 | 277.8 | 257.8 |
| (a) (Da) | | | | | | | | | |
| MW/n | | 313.9 | 257.8 | 213.7 | 246.7 | 181.6 | 147.6 | 277.8 | 257.8 |
| Molar ratio (b):(a) | | 1:2 | 1:2 | 1:2 | 1:2 | 1:2 | 1:2 | 1.2 | — |
| Polymer content | | 65.2 | 65.2 | 65.2 | 65.2 | 65.2 | 65.2 | 65.2 | 65.2 |
| (wt %) | | | | | | | | | |
| Distance between | | — | — | 0.52 | — | 0.70 | 0.25 | — | — |
| cationic groups in | | | | | | | | | |
| component (a) | | | | | | | | | |
| RESULTS | | | | | | | | | |
| IEC of resultant | | 2.33 | 2.69 | 3.06 | 2.79 | 3.48 | 4.07 | 2.52 | 2.53 |
| resin (meq/g) | | | | | | | | | |
| PS (%) of resultant | | 95 | 95 | 94 | 95 | 93 | 95 | 92 | 90 |
| membrane | | | | | | | | | |
| ER (ohm/cm²) of | | 3.2 | 1.2 | 1.6 | 1.26 | 1.66 | 1.2 | 1.1 | 1.9 |
| resultant | | | | | | | | | |
| membrane | | | | | | | | | |
| Acid stability test | | 70 | 87 | 86 | 86 | 85 | 82 | 85 | 85 |
| result for resultant | | | | | | | | | |
| membrane (%) | | | | | | | | | |

1. A cationically charged membrane obtainable from curing a composition comprising an aromatic heterocyclic compound, wherein the aromatic heterocyclic compound comprises:
a) an aromatic heterocyclic ring;
b) at least two polymerisable groups; and
c) a cationically charged nitrogen atom.

2. The cationically charged membrane according to claim 1 wherein the composition further comprises a cationically charged compound comprising only one polymerisable group.

3. The cationically charged membrane according to claim 1 wherein the cationically charged nitrogen atom forms part of the aromatic heterocyclic ring.

4. The cationically charged membrane according to claim 1 wherein the aromatic heterocyclic compound comprises two, three or four aromatic rings at least one of which is heterocyclic and at least one of which comprises a cationically charged nitrogen atom.

5. The cationically charged membrane according to claim 1 wherein the aromatic heterocyclic ring optionally further comprises an uncharged hetero-atom selected from the group consisting of N, O and S.

6. The cationically charged membrane according to claim 1 wherein the aromatic heterocyclic compound comprises at least one cationically charged pyridine, thiazole, isothiazole, pyrrole, pyrazole, imidazole, triazole, pyrimidine, pyridazine, pyrazine, oxazole, thiophene, cinnoline, quinazoline, quinoxaline, phthalazine, peteridine or carbazole ring structure and optionally a phenyl group.

7. The cationically charged membrane according to claim 1 wherein the cationically charged nitrogen is covalently bound to a methylene group.

8. The cationically charged membrane according to claim 1 further comprising an anionically charged counterion wherein the anionically charged counterion is a chloride anion.

9. The cationically charged membrane according to claim 1 wherein the aromatic heterocyclic compound is of Formula (I):
A-Z-B Formula (I)
wherein:
A and/or B are each independently selected from aromatic heterocyclic compounds of the following formulae and optionally one of A and B is an optionally substituted phenyl group: 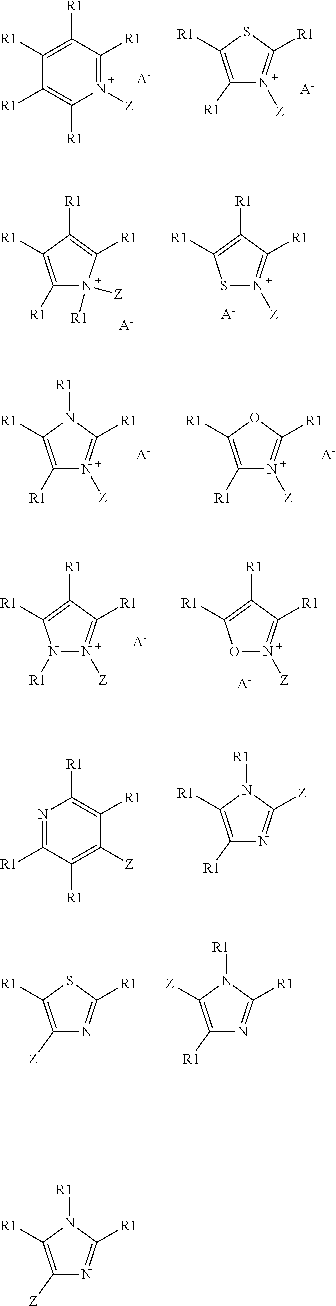
wherein:
each R1 independently is H, halogen, a polymerisable group or C₁-C₄ alkyl;
A⁻ is any negatively charged counterion; and
Z is a linking group;
provided that:
(i) at least one of A and B comprises a cationically charged nitrogen atom;
(ii) A and B each comprise at least one polymerisable group.

10. The cationically charged membrane according to claim 9 wherein A and B each comprise one and only one polymerisable group.

11. The cationically charged membrane according to claim 9 wherein Z is selected from optionally substituted C₁-alkylene; optionally substituted C₆-C₁₂ arylene, optionally substituted C₁-alkylenearylene, optionally substituted dimethylene ether, optionally substituted trimethylene amine or a combination thereof, or is a direct bond except when Z connects two charged nitrogen atoms.

12. The cationically charged membrane according to claim 1 wherein the polymerisable groups are vinyl groups.

13. The cationically charged membrane according to claim 1 wherein the aromatic heterocyclic compound comprises at least two cationically charged nitrogen atoms.

14. The cationically charged membrane according to claim 1 wherein the aromatic heterocyclic compound has a molecular weight of lower than 500 n Dalton, wherein n has a value of at least 1 and is the number of cationically charged nitrogen atoms present in the aromatic heterocyclic compound.

15. The cationically charged membrane according to claim 1 which further comprises a porous support.

16. The cationically charged membrane according to claim 1 which has an ion exchange capacity of at least 2.4 meq/g.

17. The cationically charged membrane according to claim 1 which comprises at least 1 ppm of the aromatic heterocyclic compound.

18. The cationically charged membrane according to claim 1 wherein the aromatic heterocyclic compound comprises at least two cationically charged nitrogen atoms and the distance between the at least two cationically charged nitrogen atoms is at least 0.35 nm.

19. A composition comprising:
(a) an aromatic heterocyclic compound comprising: a) an aromatic heterocyclic ring: b) at least two polymerisable groups; and c) a cationically charged nitrogen atom;
optionally (b) a cationically charged compound comprising only one polymerisable group;
optionally (c) one or more radical initiators;
optionally (d) one or more monomers free from cationically charged groups; and
optionally (e) inert solvent.

20. The composition according to claim 19 comprising
30 to 70 wt % of component (a);
0 to 40 wt % of component (b);
0 to 10 wt % of component (c);
0 to 20 wt % of component (d); and
0 to 50 wt % of component (e).

21. A process for preparing a cationically charged membrane comprising curing a composition as defined in claim 1.

22. A bipolar membrane comprising the cationically charged membrane according to claim 1.

23. A method of using the cationically charged membrane according to claim 1 for treatment of polar liquids or for the generation of electricity.

24. An electrodialysis or reverse electrodialysis unit, an electrodeionization module, a flow through capacitor, a diffusion dialysis apparatus, a membrane distillation module, an electrolyser, a redox flow battery, an acid-base flow battery or a fuel cell, comprising one or more cationically charged membranes according to claim 1.

25. The cationically charged membrane according to claim 1 wherein said membrane is an anion exchange membrane.

26. The cationically charged membrane according to claim 15 wherein said porous support is selected from the group consisting of woven and non-woven synthetic fabrics and extruded films.

27. The cationically charged membrane according to claim 1 wherein said polymerisable groups are selected from the group consisting of non-acrylic vinyl groups, allyl groups and thiol groups.

28. The cationically charged membrane according to claim 1 wherein said membrane is an anion exchange membrane;
wherein said porous support is selected from the group consisting of woven and non-woven synthetic fabrics and extruded films;
said polymerisable groups are selected from the group consisting of non-acrylic vinyl groups, allyl groups and thiol groups; and
wherein said cationically charged nitrogen is covalently bound to a methylene group.

29. A bipolar membrane comprising the cationically charged membrane according to claim 28.
